(19) United States
(12) Patent Application Publication
(10) Pub. No.: US 2025/0195911 A1
(43) Pub. Date: Jun. 19, 2025

(54) LIGHT THERAPY DEVICE STAND

(71) Applicant: Platinum IP LLC, Wilmington, DE (US)

(72) Inventor: Mark Sawyer, Lake Mary, FL (US)

(73) Assignee: Platinum IP LLC, Wilmington, DE (US)

(21) Appl. No.: 18/918,761

(22) Filed: Oct. 17, 2024

Related U.S. Application Data
(63) Continuation of application No. 18/798,360, filed on Aug. 8, 2024.
Application No. 18/798,360 is a continuation of application No. 18/643,452, filed on Apr. 23, 2024.
(60) Provisional application No. 63/612,237, filed on Dec. 19, 2023.
(60) Provisional application No. 63/612,108, filed on Dec. 19, 2023.
(60) Provisional application No. 63/612,227, filed on Dec. 19, 2023.

(51) Int. Cl. A61N 5/06

(52) U.S. Cl. CPC A61N 5/0613; A61N 5/0616; A61N 5/0617; A61N 5/0618; A61N 2005/0626; A61N 2005/0652; A61N 2005/0659; A61N 2005/0663

(57) ABSTRACT

A light therapy device stand includes a base, a motorized extension arm, a receptacle connected to the extension arm and an elongated support bar onto which one or more light therapy devices are connected. A system controller communicates wirelessly with a system remote control and a mobile application which operate the motor of the stand and each light therapy device connected to the stand. The light therapy devices are connected in one or both of a side to side configuration and a top to bottom configuration along the length of the elongated support bar, and the support bar rotates 90 degrees relative to the extension arm to transition each of the plurality of light therapy devices between a vertical orientation and a horizontal orientation.

### CROSS-REFERENCE TO RELATED APPLICATIONS

This application claims the benefit of U.S. Application Ser. No. 63/612,227 filed on Dec. 19, 2023, and U.S. patent application Ser. No. 18/643,452 filed on Apr. 23, 2024, and U.S. patent application Ser. No. 18/798,360 filed on Aug. 8, 2024 the contents of each of which are incorporated herein by reference.

### TECHNICAL FIELD

The present invention relates generally to the field of light therapy devices, and more particularly to a stand for positioning one or more light therapy devices in a specified orientation.

### BACKGROUND

The statements in this section merely provide background information related to the present disclosure and may not constitute prior art.

Scientific research has shown that exposing human skin to certain types of light can have a meaningful effect on the person's overall health. Depending on the intensity and spectrum/wavelength of the light, these effects can include reducing stress, eliminating headaches, promoting hair growth, increasing collagen production, reducing the appearance of acne, and even fat reduction.

Many commercially available light therapy devices are manufactured to include a height of about 18 inches and a width of about 8-10 inches so as to be suitable for providing light therapy to a single area of a user's body such as the head, chest, or back, for example. In this regard, it is important that a user be able to accurately position the device to ensure the desired portion of their body receives the treatment.

Most commercially available light therapy devices are provided with a mounting kit having a pair of posts that are screwed into the top surface of the device which engage a pair of elongated cords and a pulley system to allow the device to be suspended from a door or wall via a hook. However, if/when users seek to link multiple devices together side by side, they must use two separate mounting systems. When the user seeks to link multiple devices together vertically, they must connect the panels to each other using the above noted posts and keyhole openings in the bottom of each device.

Although there are known types of light therapy stands which can individually engage a light therapy device, such stands are cumbersome to use, and do not provide the ability for a user to easily maneuver one or more light therapy devices in unison to direct the light emissions in a plurality of different orientations. Additionally, such devices do not allow a user to rotate multiple light therapy devices so as to be positioned in either a horizontal or vertical manner for use when standing or lying down. Moreover, such devices require the user to physically lift the device when changing the orientation, which is difficult due to the weight of the devices and may not be possible for individuals suffering from or recovering from certain injuries.

The present invention, directed to a light therapy device stand differs from the conventional art in several aspects. The manner by which will become more apparent in the description which follows, particularly when read in conjunction with the accompanying drawings.

### SUMMARY OF THE INVENTION

The present invention is directed to a light therapy device stand. One embodiment of the present invention can include a base having a center platform, and a pair of rails extending outward therefrom. A motorized extension arm can be positioned along the top surface of the center platform and can include a plurality of telescoping sections so as to move vertically between a retracted position and an extended position. A receptacle can be positioned along the top end of the extension arm and can receive an elongated support bar onto which one or more light therapy devices can be connected.

In one embodiment, a control panel is positioned along the base for accepting user instructions to operate the motor to selectively raise and lower the height of the one or more light therapy devices connected to the support bar. In one embodiment, a system controller is provided for communicating with a system remote control device, a mobile application, and the motor. Each of the system remote control device and the mobile application can control the operation of both the stand motor and the lighting array of each light therapy device connected to the stand.

In one embodiment, the plurality of light therapy devices can be connected in one or both of a side-to-side configuration and a top to bottom configuration along the length of the elongated support bar via a combination of sliding connectors and connection rails. In one embodiment, the elongated support bar can rotate 90 degrees relative to the extension arm to transition each of the plurality of light therapy devices between a vertical orientation and a horizontal orientation.

This summary is provided merely to introduce certain concepts and not to identify key or essential features of the claimed subject matter.

### BRIEF DESCRIPTION OF THE DRAWINGS

Presently preferred embodiments are shown in the drawings. It should be appreciated, however, that the invention is not limited to the precise arrangements and instrumentalities shown.

FIG. 1 is an exploded parts view of a light therapy device stand that is useful for understanding the inventive concepts disclosed herein.

FIG. 2 is a perspective view of the base and extension arm of the light therapy device stand in the retracted position, in accordance with one embodiment of the invention.

FIG. 3 is a perspective view of the base and extension arm of the light therapy device stand in the extended position, in accordance with one embodiment of the invention.

FIG. 4A a simplified block diagram of the system controller of the stand, in accordance with one embodiment of the invention.

FIG. 4B is a front view of the remote control and mobile application of the light therapy device stand, in accordance with one embodiment of the invention.

FIG. 5A is a cutout view of the panel attachment assembly of the light therapy device stand, in accordance with one embodiment of the invention.

FIG. 5B is a side view of the light therapy device stand in operation, in accordance with one embodiment of the invention.

FIG. 5C is another side view of the light therapy device stand in operation, in accordance with one embodiment of the invention.

FIG. 5D is another side view of the light therapy device stand in operation, in accordance with one embodiment of the invention.

FIG. 5E is another side view of the light therapy device stand in operation, in accordance with one embodiment of the invention.

FIG. 5F is another side view of the light therapy device stand in operation, in accordance with one embodiment of the invention.

FIG. 6A is a cutout view of the panel attachment assembly of the light therapy device stand, in accordance with one embodiment of the invention.

FIG. 6B is a side view of the light therapy device stand in operation, in accordance with one embodiment of the invention.

FIG. 6C is another side view of the light therapy device stand in operation, in accordance with one embodiment of the invention.

FIG. 6D is another side view of the light therapy device stand in operation, in accordance with one embodiment of the invention.

FIG. 6E is another side view of the light therapy device stand in operation, in accordance with one embodiment of the invention.

FIG. 6F is another side view of the light therapy device stand in operation, in accordance with one embodiment of the invention.

### DETAILED DESCRIPTION OF THE INVENTION

While the specification concludes with claims defining the features of the invention that are regarded as novel, it is believed that the invention will be better understood from a consideration of the description in conjunction with the drawings. As required, detailed embodiments of the present invention are disclosed herein; however, it is to be understood that the disclosed embodiments are merely exemplary of the invention which can be embodied in various forms. Therefore, specific structural and functional details disclosed herein are not to be interpreted as limiting, but merely as a basis for the claims and as a representative basis for teaching one skilled in the art to variously employ the inventive arrangements in virtually any appropriately detailed structure. Further, the terms and phrases used herein are not intended to be limiting but rather to provide an understandable description of the invention.

### Definitions

As described herein, a “unit” and “section” mean a series of identified physical components which are linked together and/or function together to perform a specified function.

As described throughout this document, the term “about” “approximately” “substantially” and “generally” shall be used interchangeably to describe a feature, shape, or measurement of a component within a tolerance such as, for example, manufacturing tolerances, measurement tolerances or the like.

As described herein, the term “removably secured,” and derivatives thereof shall be used to describe a situation wherein two or more objects are joined together in a non-permanent manner so as to allow the same objects to be repeatedly joined and separated.

As described throughout this document, the term “complementary shape,” and “complementary dimension,” shall be used to describe a shape and size of a component that is identical to, or substantially identical to the shape and size of another identified component within a tolerance such as, for example, manufacturing tolerances, measurement tolerances or the like.

As described herein, the term “slidingly engage”, “telescopically connected” and derivatives thereof shall be used interchangeably to describe a situation wherein two or more identified objects are connected linearly such that one identified part slides into and out from the other identified part so as to selectively lengthen or shorten the total linear length of the assembly.

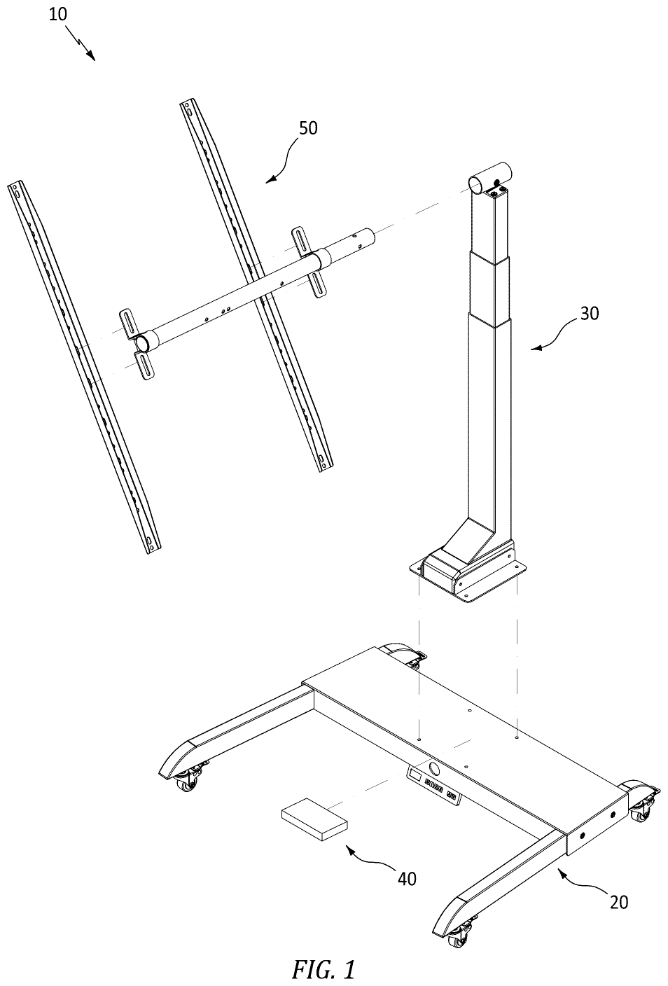

FIGS. 1-6F illustrate one embodiment of a light therapy device stand 10 that are useful for understanding the inventive concepts disclosed herein. In each of the drawings, identical reference numerals are used for like elements of the invention or elements of like function. For the sake of clarity, only those reference numerals are shown in the individual figures which are necessary for the description of the respective figure. For purposes of this description, the terms “upper,” “bottom,” “right,” “left,” “front,” “vertical,” “horizontal,” and derivatives thereof shall relate to the invention as oriented in FIG. 1.

As shown in the drawings, one embodiment of the light therapy device stand 10 can include, essentially, a base 20, an extension arm 30, a system controller 40, and a panel attachment assembly 50.

The base 20 can form the bottom end of the stand 10 and can function to provide a stable platform from which the extension arm and panel connection assembly can extend. In one embodiment, the base can include a center platform 21 having a top surface 21a, a bottom surface 21b, a front edge 21c, a back edge 21d and a pair of side edges 21e and 21f.

Additionally, the base can include a pair of rails 22 and 23 that extend outward perpendicularly from side edges 21e and 21f, respectively. Each of the rails can include single or omnidirectional casters 24 to permit the stand to be easily moved from one location to another.

As described herein, the base 20 may be formed from materials that are, for example, relatively strong and stiff for their weight. Several nonlimiting examples include but are not limited to various metals or metal alloys (e.g., aluminum, steel, titanium, or alloys thereof), plastic/polymers (e.g., high-density polyethylene (HDPE), rigid polyvinyl chloride (PVC), malleable polyethylene terephthalate (PET)), and/or various composite materials. Although described above with regard to a U-shaped base member shape, this is for illustrative purposes only, as any number of other configurations are also contemplated.

The extension arm 30 can be connected to the system controller 40 and can function to selectively raise and lower the panel assembly 50 to any number of different heights. In the preferred embodiment, the extension arm can include a lower arm segment 31, a middle arm segment 32 and an upper arm segment 33. Each of the segments can comprise elongated, generally hollow members and each of the lower arm segment 31 and the middle arm segment 32 can include openings 31a, and 32a, respectively along the upper edges for slidingly receiving an adjacent segment. More specifically, the upper arm segment 33 can be slidingly positioned within the middle arm segment 32, which itself can be slidingly positioned within the lower arm segment 31.

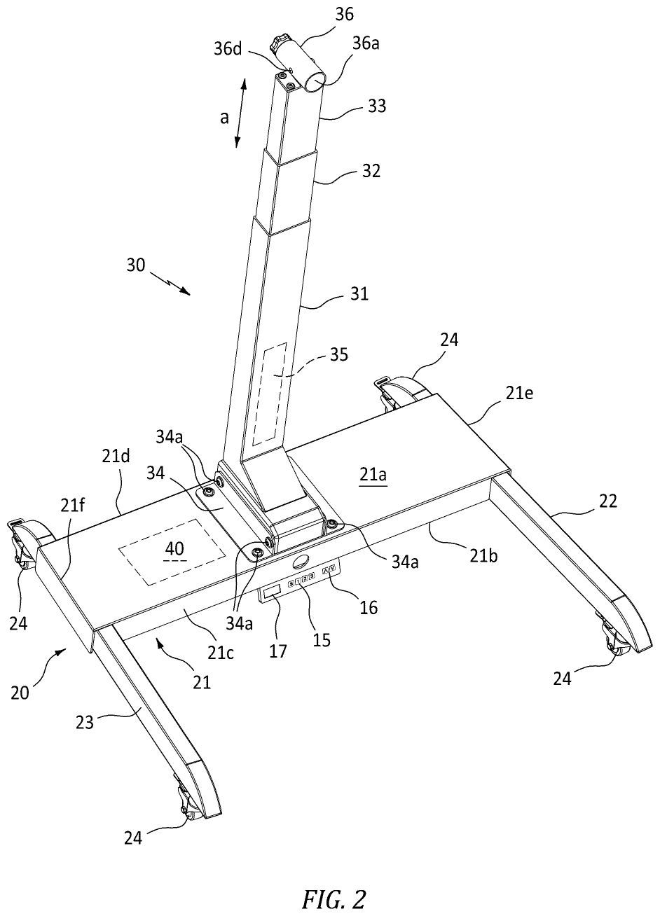

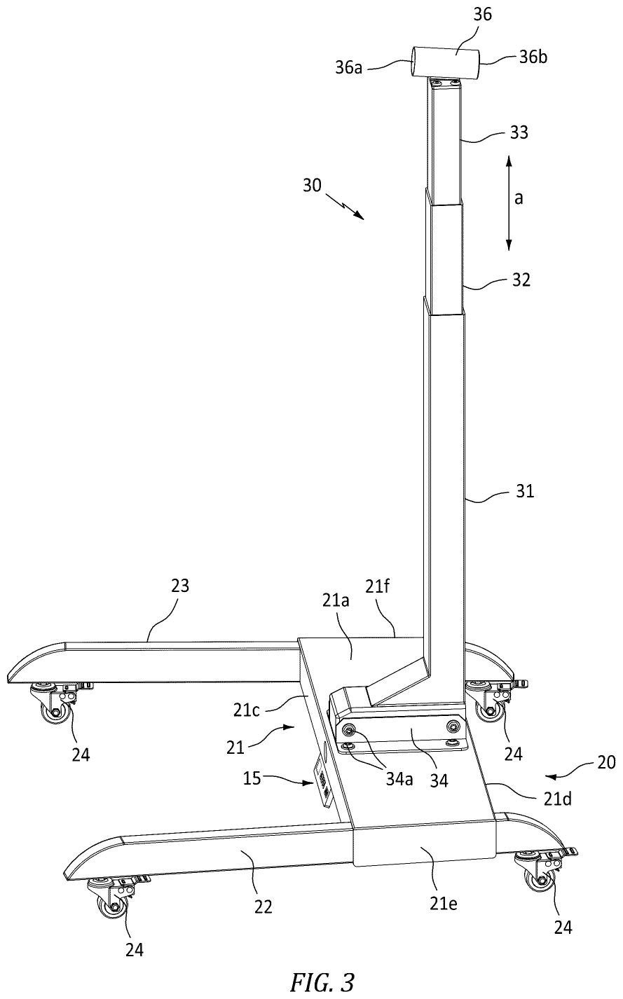

In the preferred embodiment, the extension arm 30 can be secured along the top surface of the center platform via a mounting bracket 34 and mounting hardware 34a such as screws or bolts, for example. Likewise, a motor 35 such as a linear actuator, for example, can be positioned within the lower arm segment 31. As shown by arrow a, the motor can function to extend and retract arm segments 32 and 33 to position the same anywhere between the fully retracted position shown at FIG. 2, and the fully extended position shown at FIG. 3.

In one embodiment, a receptacle 36 is positioned along the top end of the sliding arm section 33. As shown the receptacle can include a generally hollow and cylindrical-shaped member having an open first end 36a, a closed back end 36b, a back opening 36c and a side opening 36d along the sidewall. The hollow interior space formed by the receptacle will include a shape and size that is complementary to the shape and size of one end of the below described support bar 51 so as to removably and rotatably engage bar to orient the light therapy devices in any number of user-desired manners.

In either instance, each of the arm segments 31, 32 and 33 may also be formed from materials that are, for example, relatively strong and stiff for their weight. Several nonlimiting examples include but are not limited to various metals or metal alloys, various plastics/polymers, and/or various composite materials. Additionally, although described above with regard to three arm segments and a linear-type motor, this is for illustrative purposes only. To this end, other embodiments having a different number of arm segments, and/or any number of other types of electrically actuated motors capable of selectively engaging one or both of the middle arm segment and the upper arm segment to perform the stated functionality are also contemplated.

The stand 10 can include a control panel 15 that is communicatively linked to the system controller 40 in order to allow a user to control the operation of the extension arm 30. In this regard, the control panel can include any number of physical buttons 16 and/or display screens 17. In some embodiments, the display screen can include, comprise, or consist of a Graphic User Interface (GUI) touch screen device for conducting two-way communication with a user. To this end, the interface can allow the user to instruct the system to perform functions such as raising or lowering the extension arm to any number of user-defined heights.

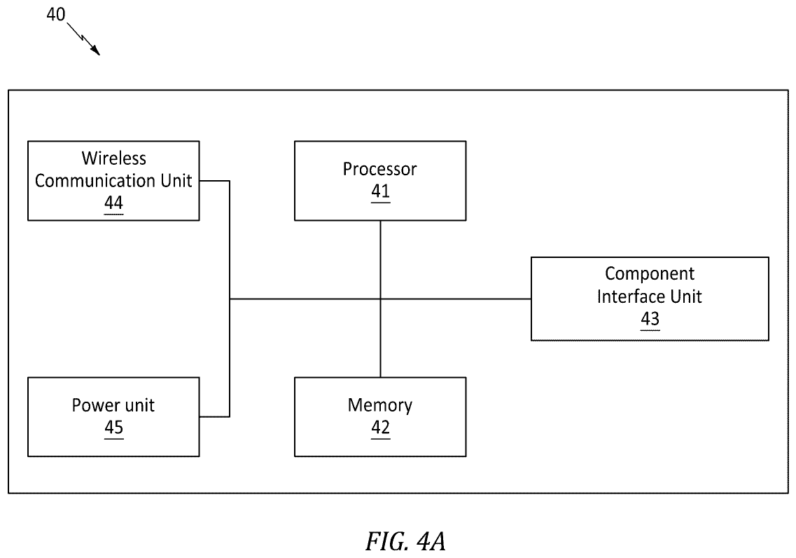

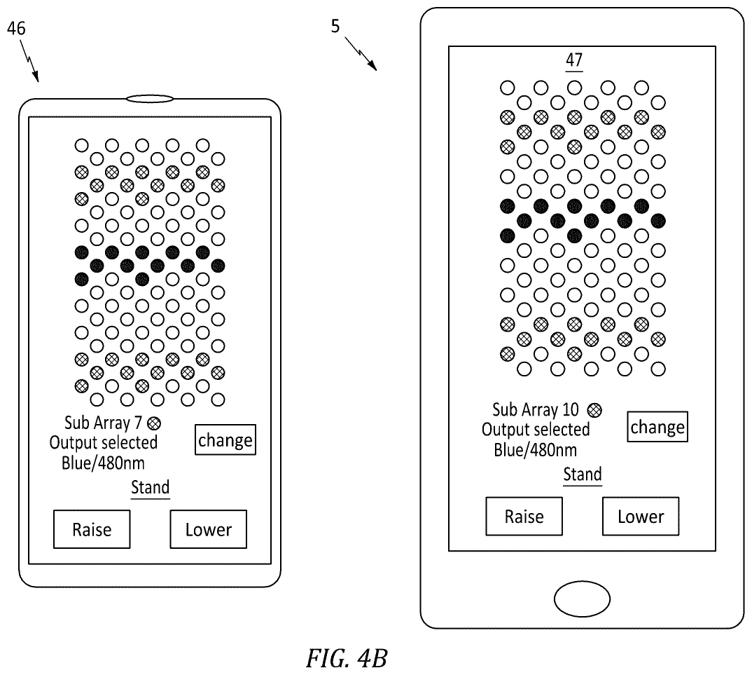

FIG. 4A is a simplistic block diagram illustrating one embodiment of the controller 40 that includes a processing unit 41 that is conventionally connected to an internal memory 42, a component interface unit 43, a wireless communication unit 44, and/or a power unit 45.

Although illustrated as separate elements, those of skill in the art will recognize that one or more system components may comprise or include one or more printed circuit boards (PCB) containing any number of integrated circuit or circuits for completing the activities described herein. The CPU may be one or more integrated circuits having firmware for causing the circuitry to complete the activities described herein. Of course, any number of other analog and/or digital components capable of performing the described functionality can be provided in place of, or in conjunction with the described elements.

The processing unit 41 can include one or more central processing units (CPU) or any other type of device, or multiple devices, capable of manipulating or processing information such as program code stored in the memory 42 in order to allow the device to perform the functionality described herein.

Memory 42 can act to store operating instructions in the form of program code for the processing unit 41 to execute. Although illustrated in FIG. 4 as a single component, memory 42 can include one or more physical memory devices such as, for example, local memory and/or one or more bulk storage devices. As used herein, local memory can refer to random access memory or other non-persistent memory device(s) generally used during actual execution of program code, whereas a bulk storage device can be implemented as a persistent data storage device such as a hard drive, for example. The bulk storage device can contain any number of different programs that permit the processor to perform the functionality described herein and can also receive and store the exercise information for each user.

The component interface unit 43 can function to provide a communicative link between the processing unit 41, the extension arm motor 35, and the control panel 15. In this regard, the component interface unit can include any number of different components such as one or more PIC microcontrollers, standard bus, internal bus, connection cables, wireless receiver and/or associated hardware such as USB cables and connectors, and other such hardware capable of linking the various components. Of course, any other means for providing the two-way communication between the system components can also be utilized herein.

The communication unit 44 can include any number of components capable of sending and/or receiving electronic signals with another device, either directly or over a network. In the preferred embodiment, the communication unit 44 can include a Wi-Fi transceiver for communicating directly with one or both of a system coded remote-control device 46 and/or a user device such as a smartphone 5 or tablet running a mobile application 47, for example as shown at FIG. 4B.

In one embodiment, the light therapy device 1, the remote control 46 and the mobile application 47 can comprise the light therapy device, system remote control and mobile application described in copending U.S. patent application Ser. No. 18/643,452 to Sawyer, the contents of which are incorporated herein by reference. In this regard, the remote control 46 and mobile application 47 can function to control both the stand, and any number of light devices secured onto the stand via a single device.

Of course, any number of other transmission and reception mechanisms and protocols can also be utilized herein, several nonlimiting examples include cellular transceivers, Near-Field-Communication (NFC) devices, radio, infrared (IR), RFID, and/or network adapter(s) functioning to communicate over a WAN, LAN, or the internet via an internet service provider, among others, for example.

The power unit 45 can include any number of different components capable of providing the necessary power requirements to each element of the device. In the preferred embodiment, the power unit can include or comprise an A/C electrical power transformer and electrical cord for allowing the device to be plugged into a standard electrical outlet. Of course, other embodiments are contemplated wherein the power unit includes onboard batteries so as to operate without the need to connect to a power outlet.

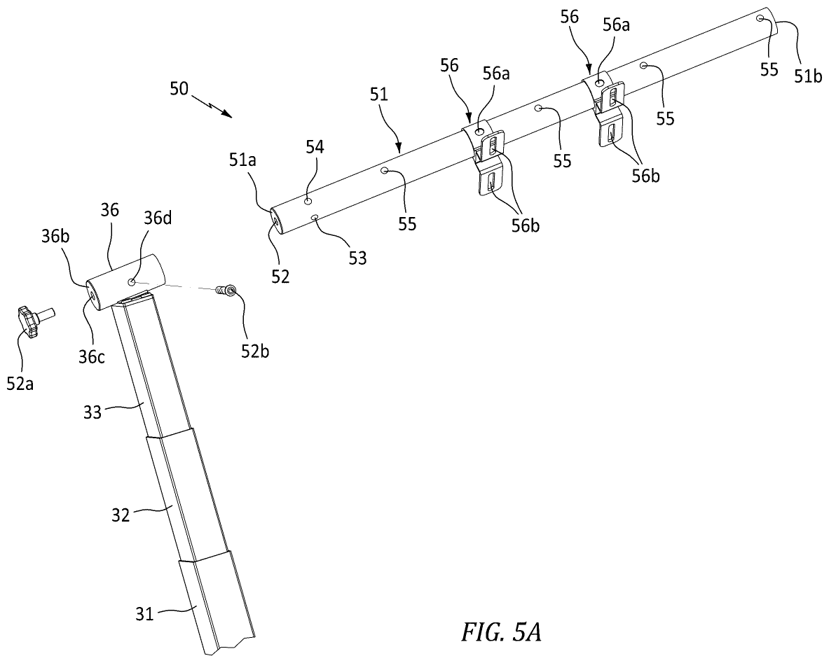

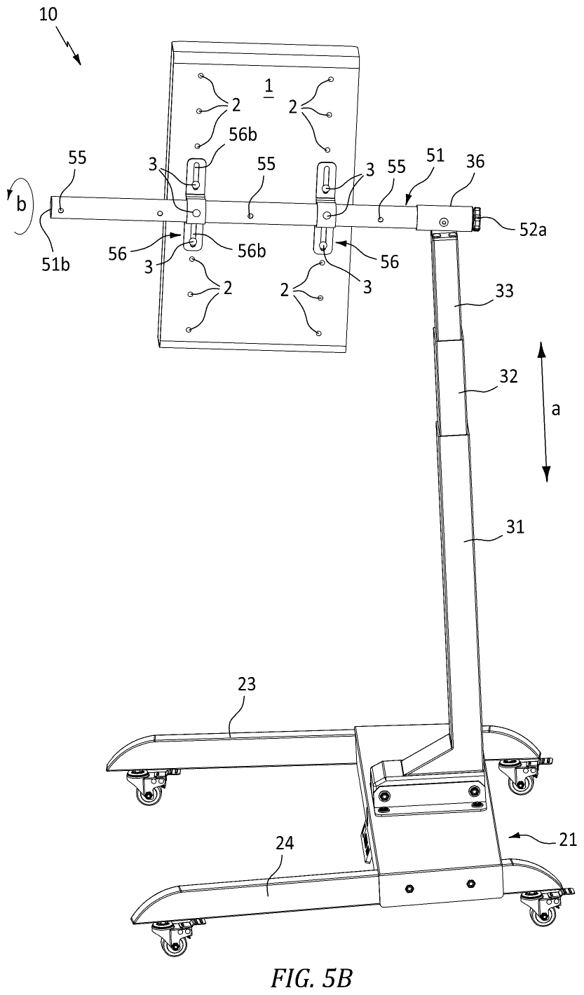

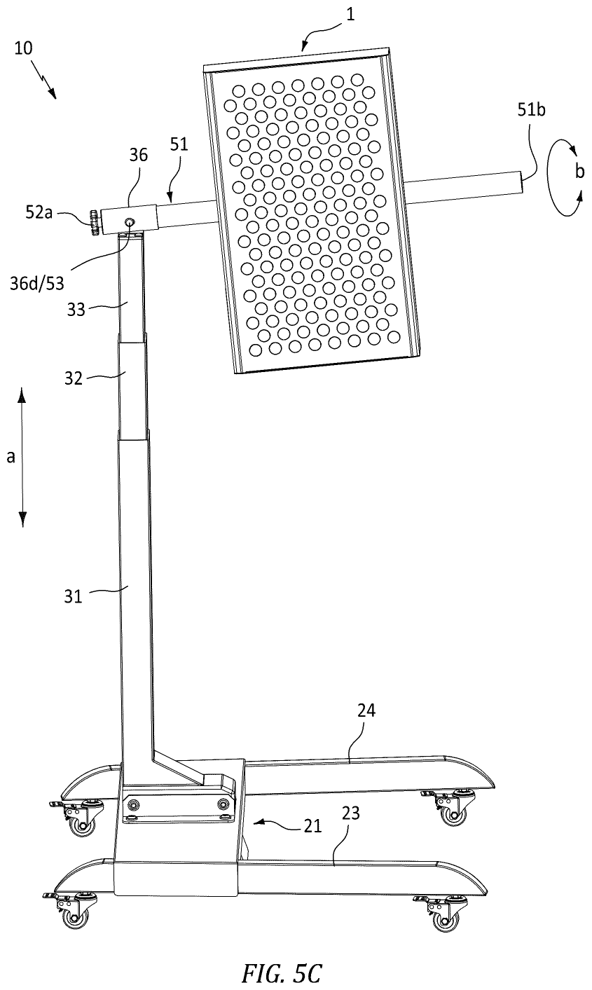

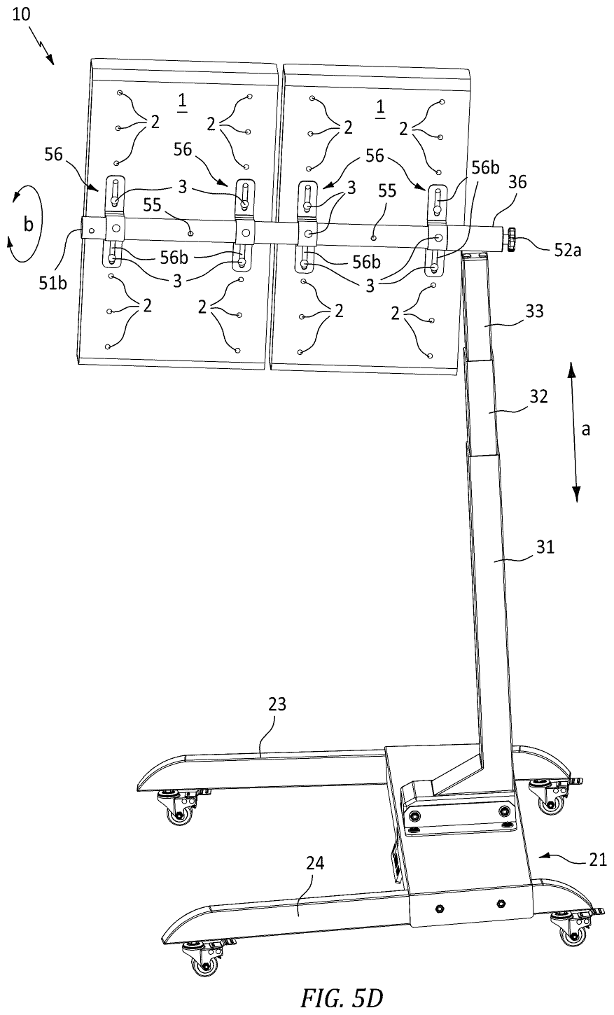

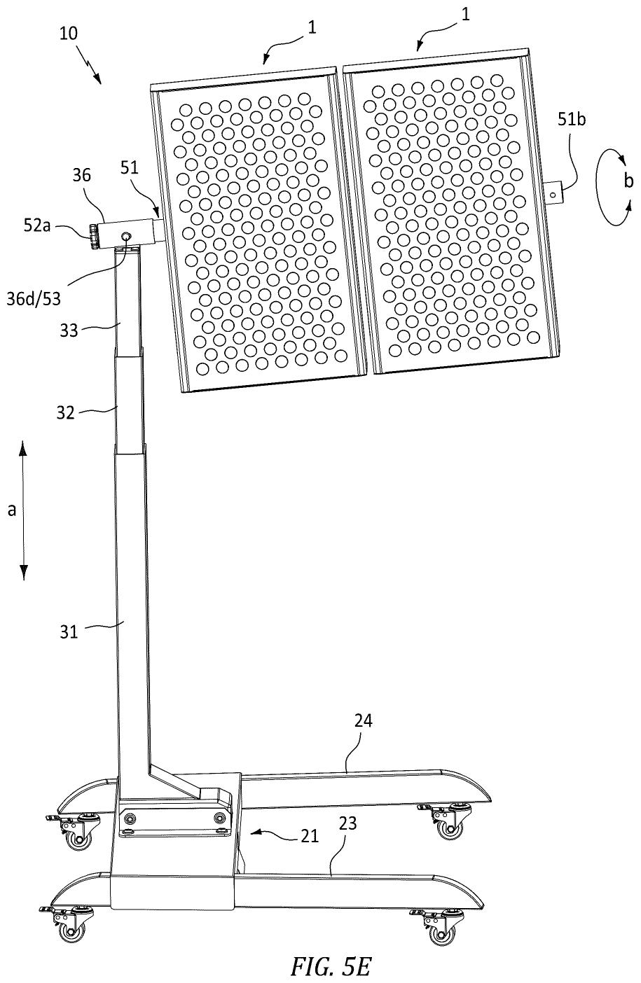

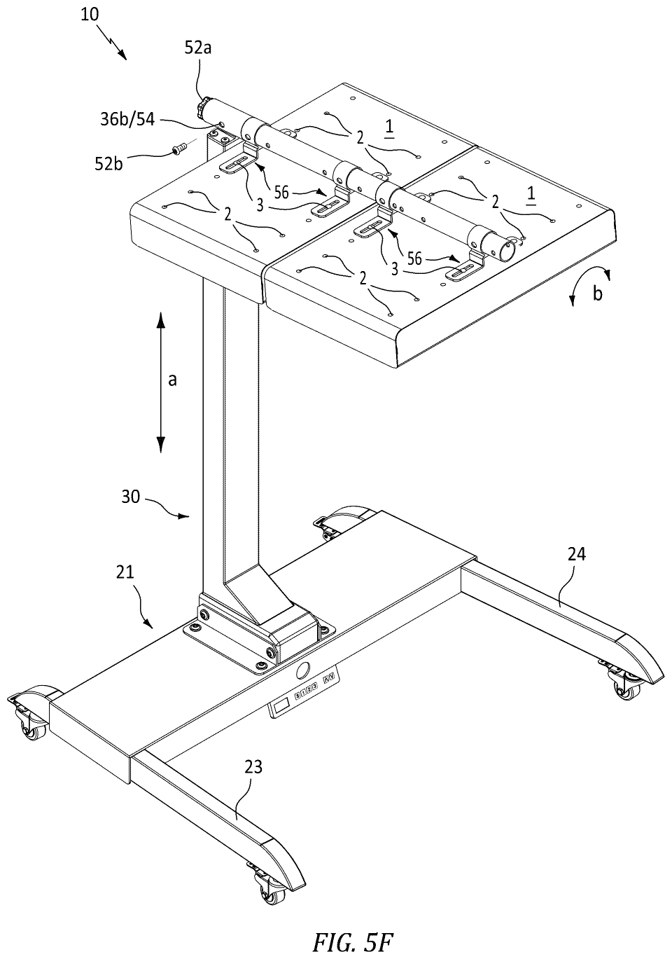

FIGS. 5A-5F illustrate one embodiment of the panel attachment assembly 50. As shown best at FIG. 5A, the assembly can include an elongated support bar 51 having a first end 51a, a second end 51b, a lateral threaded opening 52, a vertical-positioning threaded opening 53 a horizontal-positioning threaded opening 54 and a plurality of sliding panel threaded openings 55 that are aligned serially with each other and the horizontal-positioning threaded opening 54.

In the preferred embodiment, the bar 51 can be constructed from a solid rigid material such as aluminum or steel round bar, for example, however other shapes and construction materials are also contemplated.

In one embodiment, a plurality of sliding connectors 56 can be provided for securing one or more light therapy devices 1 onto the bar for use with the stand. Each of the sliding connectors can include a generally horseshoe-shaped member having a cylindrical-shaped center section and two serially aligned connection arms extending outward therefrom along an identical plane. As described herein, the cylindrical central section of each connector 56 can include a shape and size that is complementary to the cross-sectional shape and size of the bar 51 and can function to slide along the length thereof.

In one embodiment, each of the sliding connectors 56 can include a center opening 56a that is positioned along the central section of the connector. The center opening can be aligned with one of the plurality of threaded apertures 55 along the length of the support bar 51 and can function to receive mounting hardware such as a bolt, for example, that passes through the opening 56a and engages the aperture 55 to secure the connector to the bar.

In one embodiment, each of the sliding connectors 56 can also include elongated slotted openings 56b along the connection arms. Each of the slotted openings 56b are spaced at locations complementary to the locations of the mounting apertures 2 located along the back surface of a light therapy device 1. In this regard, additional mounting hardware such as a bolt 3, for example, can pass through the openings 56b to engage the mounting apertures 2 on the light therapy device 1 to secure the device onto the sliding connector.

As noted above, the support bar 51 is removably and rotatably secured onto the extension arm 30 via the receptacle 36. As such, the lateral threaded opening 52 is positioned along the end of the bar 51a so as to be aligned with the back opening 36c when the bar is positioned within the receptacle 36. When so positioned, a first thumbscrew 52a or other such hardware can pass through the opening 36c to engage the lateral threaded opening 52 in order to secure the bar onto the extension arm and to prevent lateral separation of the same.

The vertical-positioning threaded opening 53 is positioned at a 90° angle relative to the horizontal-positioning threaded opening 54, and both openings include a separation distance from the first end 51a that is complementary to the separation distance at which the side opening 36d is positioned relative to the back opening 36c on the receptacle 36. Both threaded openings can be selectively engaged by a second thumbscrew 52b or other such hardware that can pass through the side opening of the receptacle.

As such, when the first end 51a of the bar is positioned within the receptacle 36, the bar 51 can rotate (see arrow b) 90° to selectively align either the vertical-positioning threaded opening 53 or the horizontal-positioning threaded opening 54 with the side opening 36d of the receptacle 36 to position the light therapy panels in a vertical or horizontal orientation as shown respectively at FIGS. 5E and 5F. Regardless of the orientation, operation of the stand motor allows a user to selectively rise and lower the array of light therapy devices to any number of desired heights as shown by arrow a.

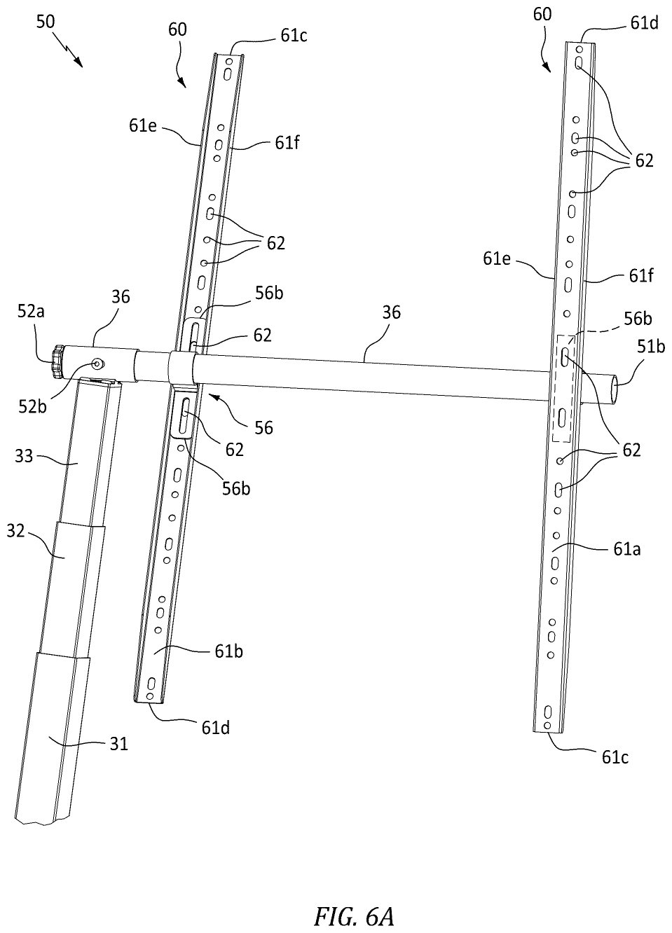

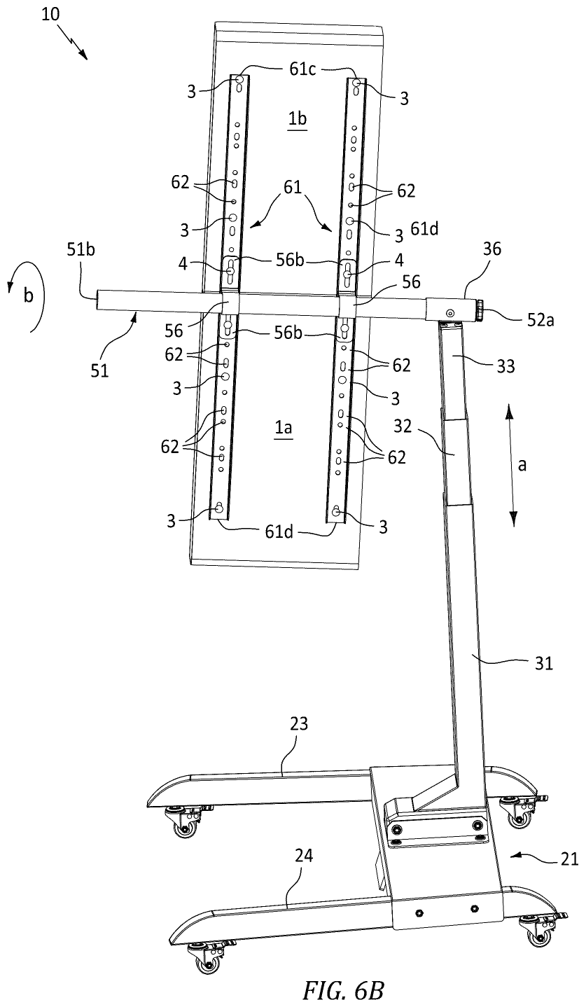

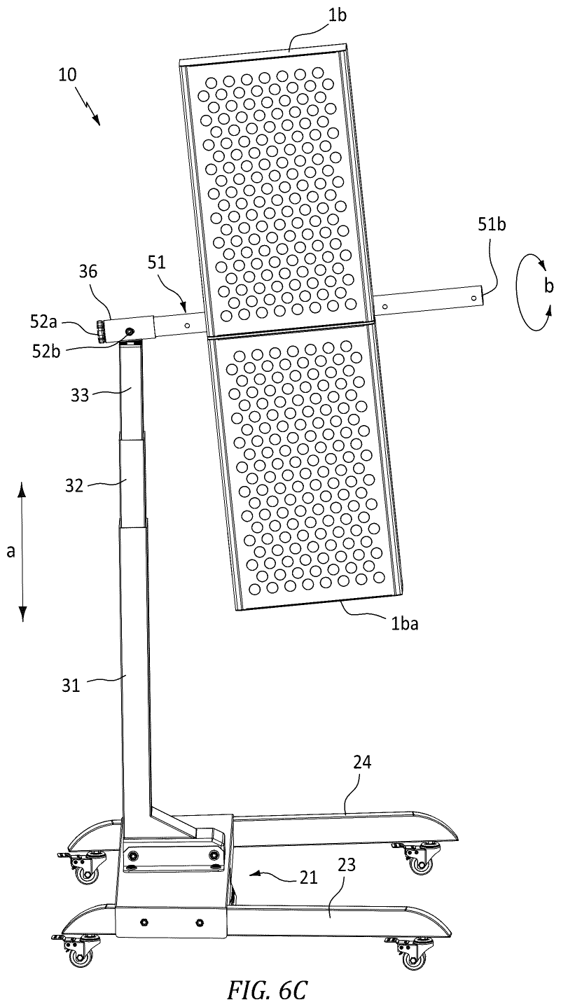

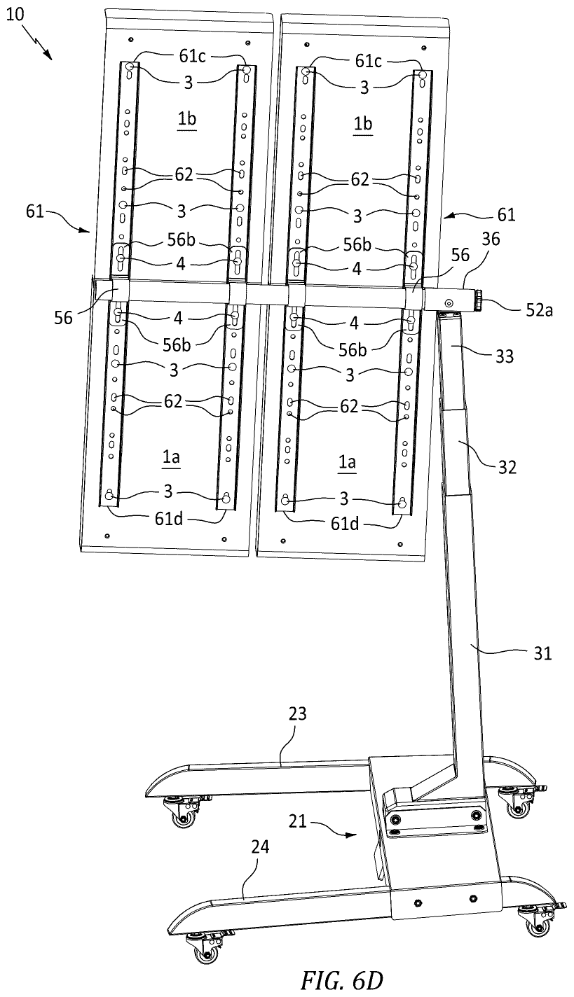

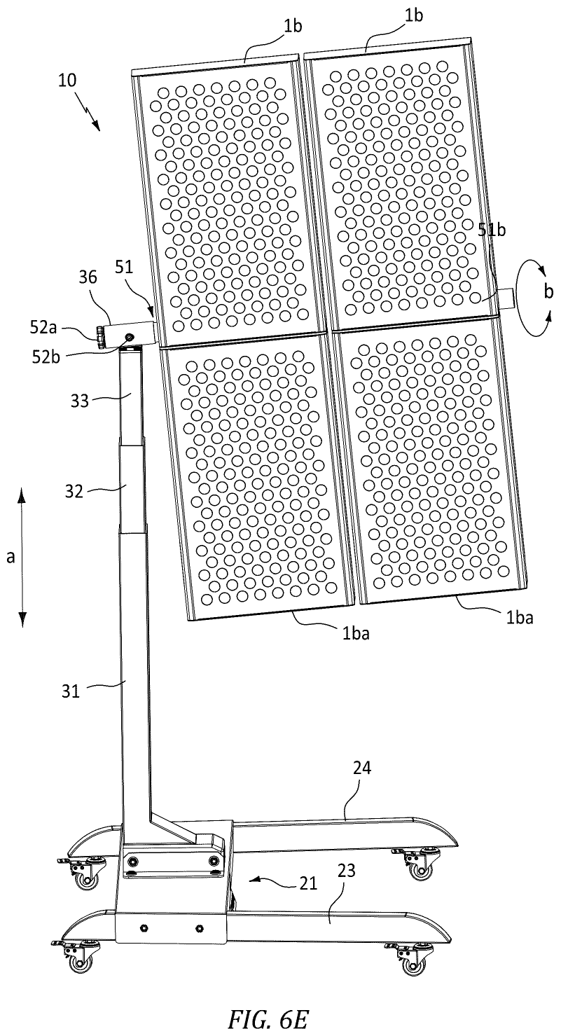

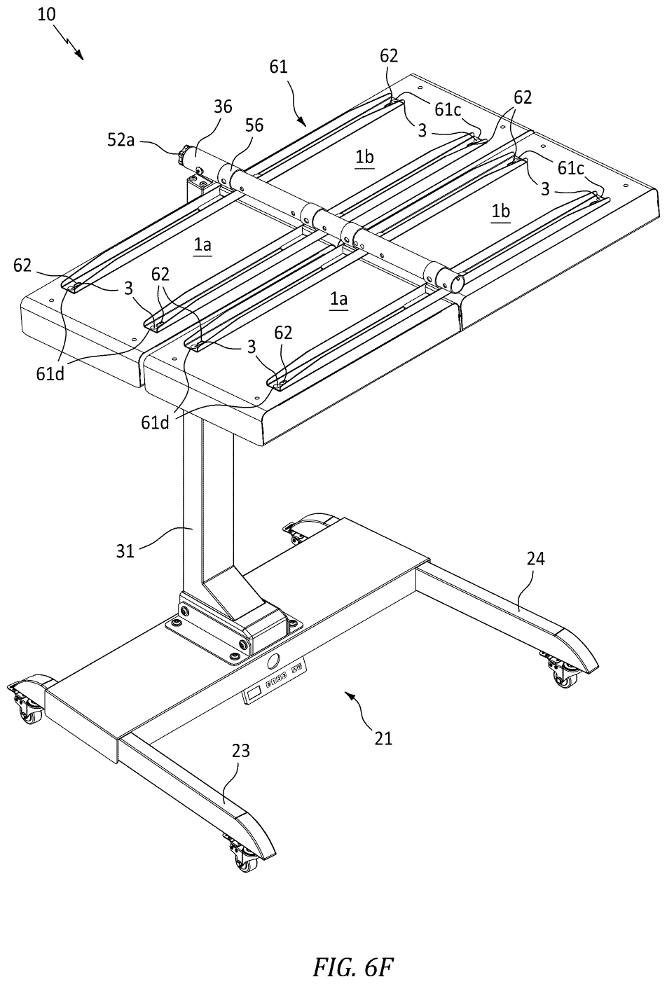

As shown at FIGS. 6A-6D, one embodiment of the panel attachment assembly 50 can include a plurality of connection rails 60 that function to allow any number of light therapy devices to be secured onto the stand in both a side by side configuration and in a top to bottom configuration (e.g., stacked configuration). As shown best at FIG. 6A, each connection rail 60 can include an elongated member having a flat front surface 61a, a flat back surface 61b, a first end 61c, a second end 61d and a pair of side rails 61e and 61f that extend orthogonally outward from the back surface. Each of the rails can also include a plurality of openings 62 that are spaced along the length of the rails so as to be complementary to the locations of the mounting apertures 2 located along the back surface of a light therapy device 1.

In one embodiment, the side rails 61e and 61f can include a separation distance that is complementary to the width of the sliding connector 56, so as to position the arms of the connector against the back surface 61b. When so positioned, the slotted openings 56b will be aligned with at least two of the openings 62 on the rail.

In operation, connector hardware such as elongated bolts 4 can pass through the slotted openings 56b and the respectively aligned opening 62 of the rail to engage mounting apertures 2 located on the top end of one light therapy device 1a, and the bottom end of a second light therapy device 1b that are positioned in the stacked configuration. When so positioned, the remaining openings 62 along the rail will be aligned with the other mounting apertures 2 on each light therapy device and can be engaged by mounting hardware 3 such as bolts, for example.

Moreover, when in the stacked configuration, the support bar 51 can be rotated (see arrow b) to position the light therapy devices in either a vertical orientation, or a horizontal orientation, as shown. Regardless of the configuration and orientation, operation of the stand motor allows the user to selectively rise and lower the array of devices to any number of desired heights as shown by arrow a.

In the preferred embodiment, the connection rails will include a length (e.g., distance between first end 61c and second end 61d) of about 38 inches. Such a dimension is important to allow the rails to be connected along the full length (from the bottom end to the top end) of two standard sized light therapy devices 1a and 1b such as the BioMax 300 or BioMax 450 light therapy devices that are commercially available from Platinum LED Therapy Lights®, in a stacked configuration, and each having a length of about 19 inches. Such a feature prevents the devices from moving relative to each other which could result in uneven alignment with a patient's body. Of course, the rails can be manufactured in other dimensions so as to engage two stacked light therapy devices having different dimensions.

Accordingly, the inventive stand 10 can allow a user to securely position any number of individual light therapy devices individually, in a side-by-side configuration and/or in a stacked configuration, and to orient the same anywhere between a purely horizontal and purely vertical position. Such a feature permits the inventive stand 10 to position one or more light therapy devices for use by a user when standing (with the devices positioned vertically) or when laying on a treatment table (with the light therapy devices positioned horizontally). Moreover, such a feature allows users to easily move the entire system from one location to another without removing, disconnecting, or adjusting the position of the light therapy devices individually.

As to a further description of the manner and use of the present invention, the same should be apparent from the above description. Accordingly, no further discussion relating to the manner of usage and operation will be provided.

As described herein, one or more elements of the light therapy device stand 10 can be secured together utilizing any number of known attachment means such as, for example, screws, glue, compression fittings and welds, among others. Moreover, although the above embodiments have been described as including separate individual elements, the inventive concepts disclosed herein are not so limiting. To this end, one of skill in the art will recognize that one or more individually identified elements may be formed together as one or more continuous elements, either through manufacturing processes, such as welding, casting, or molding, or through the use of a singular piece of material milled or machined with the aforementioned components forming identifiable sections thereof.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Likewise, the term “consisting” shall be used to describe only those components identified. In each instance where a device comprises certain elements, it will inherently consist of each of those identified elements as well.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. A light therapy device stand, said stand comprising:
a base having center platform, and a pair of rails extending outward therefrom;
an extension arm having a lower arm segment that is connected to a top surface of the center platform, and an upper arm segment;
a receptacle that is positioned along a top surface of the upper arm segment;
an elongated support bar that is configured to be connected to the receptacle; and
at least one connector that is configured to engage each of the elongated support bar and a back surface of a light therapy device.

2. The stand of claim 1, wherein the extension arm is configured to transition between an extended position and a retracted position.

3. The stand of claim 2, wherein in the extended position the light therapy device is moved away from the base, and in the retracted position the light therapy device is moved toward the base.

4. The stand of claim 2, further comprising:
a motor that is connected to the extension arm.

5. The stand of claim 4, wherein the motor is configured to selectively transition the extension arm between the extended position and the retracted position.

6. The stand of claim 5, further comprising:
a control panel that is in communication with the motor.

7. The stand of claim 6, wherein the control panel is configured to receive a user input to instruct an operation of the motor.

8. The stand of claim 1, further comprising:
a plurality of casters that are located along the pair of rails.

9. The stand of claim 1, wherein the receptacle includes an open first end, and the elongated support bar includes a first end that is removably positioned within the open first end of the receptacle.

10. The stand of claim 1, further comprising:
a middle arm segment that is positioned between the lower arm segment and the upper arm segment, and
wherein each of the lower arm segment, the middle arm segment and the upper arm segment are telescopically connected.
